(19) United States
(12) Patent Application Publication
(10) Pub. No.: US 2022/0183996 A1
(43) Pub. Date: Jun. 16, 2022

(54) High-Efficiency Transdermal Patches

(71) Applicant: Global Biomedical Technologies, LLC, Naples, FL (US)

(72) Inventors: Howard S. Rosing, SR., Naples, FL (US); Yadong Wang, Ithaca, NY (US)

(21) Appl. No.: 17/689,506

(22) Filed: Mar. 8, 2022

Related U.S. Application Data
(63) Continuation of application No. 16/354,838, filed on Mar. 15, 2019, now abandoned.
Application No. 16/354,838 is a continuation of application No. 15/939,143, filed on Mar. 28, 2018, now abandoned.
(60) Provisional application No. 62/477,971, filed on Mar. 28, 2017.

(51) Int. Cl. A61K 9/70; A61K 45/06; A61K 47/34; A61K 31/00

(52) U.S. Cl. CPC A61K 9/7061; A61K 31/00; A61K 47/34; A61K 45/06

(57) ABSTRACT

In one embodiment, a drug is administered to a subject using a transdermal patch that includes a substrate and a layer of pressure-sensitive adhesive provided on the substrate, the pressure-sensitive adhesive comprising a blend of an adhesive compound and a carrier-drug compound, the carrier-drug compound comprising a drug transport compound and a drug that is to be delivered to the skin of the subject, wherein the drug transport compound transports the drug through the pressure-sensitive adhesive to the skin.

### CROSS-REFERENCE TO RELATED APPLICATION

This application is a continuation of copending U.S. Non-Provisional application having Ser. No. 16/354,838, entitled “High Efficiency Transdermal Patches,” filed Mar. 15, 2019, which claims priority to, and the benefit of, U.S. Non-Provisional application having Ser. No. 15/939,143, filed Mar. 28, 2018, which claims priority to, and the benefit of, U.S. Provisional Application Ser. No. 62/477,971, filed Mar. 28, 2017, all of which are hereby incorporated by reference herein in their entireties.

### BACKGROUND

Transdermal patches are often used to deliver drugs to individuals. Such patches often comprise a medicated adhesive that is placed in contact with the skin to deliver a specific dose of medication to the individual. One advantage of transdermal drug delivery over other types of medication delivery such as oral, topical, intravenous, intramuscular, etc., is that the patch provides controlled release of the medication to the patient. One disadvantage of transdermal drug delivery, however, is that it tends to be inefficient in terms of its drug delivery. Specifically, typically only a fraction of the drug that is contained within a transdermal patch can actually be administered to the individual by the patch as much of the drug is retained within the patch. Because of this, transdermal patch manufacturers must use a larger amount of a drug to manufacture the patch than the amount that is required for dosing. In such cases, significant amounts of residual drug are unused and are, therefore, wasted. While this may not create a concern when the drug is inexpensive, it can be a significant concern when the drug is expensive to produce or obtain. Moreover, in situations in which the residual drug has a significant potential for abuse, as in the case of opioids, it is undesirable for substantial amounts of residual drug to exist irrespective of its cost.

In view of the above discussion, it can be appreciated that it would be desirable to have a more efficient transdermal patch with which a higher percentage of drug contained within the patch can actually be administered to the individual.

### BRIEF DESCRIPTION OF THE DRAWINGS

The present disclosure can be better understood with reference to the following drawings. The components in the drawings are not necessarily to scale.

FIG. 1 is a side view of an embodiment of a transdermal patch that can be used to deliver a dose of medication to an individual through the skin.

FIG. 2 is a partial detail view of a first configuration for the transdermal patch of FIG. 1.

FIGS. 3A and 3B are partial detail views of a second configuration for the transdermal patch of FIG. 1, showing the patch before and after application of an accumulation solution.

### DETAILED DESCRIPTION

As described above, existing transdermal patches are inefficient in terms of drug delivery because much of the drug contained within the patch is retained within the patch and, therefore, is unavailable for administration to the individual (e.g., patient). It would, therefore, be desirable to have more efficient transdermal patches in which a higher percentage of the drug contained within the patch can actually be administered to the individual. In such cases, bioavailability is increased, smaller quantities of drug would be needed to manufacture the patch for the same dosage, and the potential for abuse of residual drug would be reduced. Described herein are examples of such transdermal patches. In one embodiment, a transdermal patch comprises a substrate that supports a pressure-sensitive adhesive that contains a drug transport compound that transports a drug that is to be administered to an individual to the skin of the individual. When such a drug transport compound is provided, a greater percentage of the drug contained in the patch can be administered to the individual, thereby reducing waste and the opportunity for abuse.

In the following disclosure, various specific embodiments are described. It is to be understood that those embodiments are example implementations of the disclosed inventions and that alternative embodiments are possible. All such embodiments are intended to fall within the scope of this disclosure.

The disclosed transdermal patches comprise a pressure-sensitive adhesive including an adhesive compound that is blended with a drug transport compound, which is configured to transport one or more drugs to the surface of the pressure-sensitive adhesive adjacent the skin. These two compounds are immiscible with each other, which facilitates the drug transport. In some embodiments, the adhesive compound comprises an acrylic polymer adhesive. In other embodiments, the adhesive compound comprises a different adhesive, such as silicone adhesive, rubber adhesive, polyurethane adhesive, or hydrocolloid blended with an adhesive such as polyisobutylene or styrene-soprene-styrene. In cases in which the adhesive compound is an acrylic polymer adhesive, the adhesive compound can be made from the polymerization of acrylic acid with variations in the chemical composition designed to balance internal cohesion or shear, as well as tack and peel strength. Irrespective of its composition, the adhesive compound provides the adhesive strength to the pressure-sensitive adhesive that enables it to stick to human and animal skin.

In some embodiments, the drug transport compound comprises a hydrophobic prepolymer formed from a multifunctional alcohol and a multifunctional carboxylic acid. As used herein, the term “prepolymer” describes an uncured polymeric mixture that exhibits little or no crosslinking. Although the cured and cross-linked polymer could be used as a drug transport compound, it is undesirable as such because it would be much less efficient than the prepolymer in that capacity. The term “multifunctional alcohol” refers to any alcohol that has two or more hydroxyl (—OH) groups, while the term “multifunctional carboxylic acid” refers to any carboxylic acid that has two or more acid (—COOH) groups. Example multifunctional alcohols include glycerol, monomeric carbohydrates such as glucose and mannose, and small polyols such as oligo (vinyl alcohol). Example multifunctional carboxylic acids include diacids such as sebacic acid, succinic acid, oxalic acid, and malic acid, and triacids such as citric acid. In some embodiments, the drug transport compound comprises a prepolymer comprising a mixture of glycol and sebacic acid, which is referred to herein as glycerol-sebacic acid prepolymer. An example of synthesis of glycerol-sebacic acid prepolymer is described below.

Highly-purified sebacic acid can be used to prepare the glycerol-sebacic acid prepolymer. In some embodiments, sebacic acid can be rigorously purified by combining a relatively small amount of sebacic acid with a relatively large amount of ethanol and heating the mixture until the sebacic acid completely dissolves. Once the sebacic acid has dissolved, the hot sebacic acid solution can be filtered under a vacuum and the filtrate can be refrigerated for several hours to enable crystallization. The sebacic acid crystals can then be collected and intermittently filtered under vacuum to collect the crystals. After the completion of the filtration, the above process (dissolution, crystallization, and filtration) can be repeated multiple times (e.g., 3-4 times) to ensure a high level of purification. Thereafter, the air-dried sebacic acid crystals can be heated under a vacuum to remove any residual ethanol or moisture.

In some embodiments, the glycerol-sebacic acid prepolymer can be prepared by mixing glycerol and sebacic acid at a molar ratio of approximately 0.5 to 1.5 glycerol to 1 sebacic acid, heating the mixture at an elevated temperature of approximately 120 to 150° C. until water is generated under nitrogen atmosphere, and maintaining the reaction at the elevated temperature as the pressure is reduced until an approximately 0.5 to 2 molar equivalent of water is removed from the mixture. In some embodiments, the reaction can be performed under a vacuum of approximately 1 to 5 Torr. The glycerol-sebacic acid prepolymer can then be permitted to cool and solidify.

The pressure-sensitive adhesive can be formed by blending the drug transport compound (e.g., glycerol-sebacic acid prepolymer) with the adhesive compound (e.g., acrylic polymer adhesive). Prior to doing so, however, the drug or drugs to be delivered can be blended with the drug transport compound. In some embodiments, the one or more drugs are hydrophobic drugs. Example hydrophobic drugs that can be dissolved with the drug transport compound include fentanyl, buprenorphine, aspirin, acetaminophen, lidocaine, bupivacaine, nitroglycerin, paclitaxel, estradiol, ethinyl estradiol, estrogen, nicotine, clonidine, fentanyl, fentanyl hcl, scopolamine, testosterone, epinephrine, norethindrone, norelgestromin, levonorgestrel, oxybutynin, tetracaine, methylphenidate, selegiline, rotigotine, rivastigmine, menthol, methyl salicylate, methyphenidate transdermal vitamin B12, 5-Hydroxytryptophan, lorazepam, diphenhydramine, haloperidol, sumatriptan, stilboestrol, sufentanil, and paclitaxel.

When the drug transport compound is a solid at room temperature, as is the case for glycerol-sebacic acid prepolymer, it can be melted, or dissolved with a solvent to facilitate blending with the drug(s). In such a case, the liquefied drug transport compound can act as a solvent for the drug(s). When melting is to be performed, the drug transport compound can be melted at a temperature of approximately 40 to 80° C. When the drug transport compound is to be dissolved, a polar organic solvent can be used. Example solvents include ethyl acetate, ethanol, and tetrahydrofuran. In some embodiments, the solvent can be added to the drug transport compound in a concentration of approximately 10 to 90 w/w %.

Glycerol-sebacic acid prepolymer contains hydrophobic domains and can form non-covalent interactions with hydrophobic drug molecules. As such, the glycerol-sebacic acid prepolymer is an inexpensive, water insoluble, biodegradable, biocompatible prepolymer that becomes a carrier for hydrophobic drugs. Glycerol-sebacic acid prepolymer is colloidally stable in aqueous media for prolonged periods of time and the low critical aggregation concentration (CAC) of glycerol-sebacic acid prepolymer implies that the glycerol-sebacic acid prepolymer is unlikely to rapidly disassemble within the adhesive compound.

It is noted that, in some embodiments, glycerol-sebacic acid prepolymer nanoparticles can be formed prior to blending with the one or more drugs. Because glycerol-sebacic acid prepolymer is not water soluble, it still exhibits hydrophobic behavior in aqueous solutions. Stable glycerol-sebacic acid prepolymer nanoparticles can be obtained using a simple solvent displacement method in water by creating a colloidal suspension between two separate phases. Briefly, a concentrated glycerol-sebacic acid prepolymer solution in ethyl alcohol is diluted into deionized water through simple stirring, with a fine dispersion being formed. The hydrophobic interior of the glycerol-sebacic acid prepolymer nanoparticles accommodate other hydrophobic compounds that that might otherwise precipitate from solution.

Once the drug or drugs have been blended with the drug transport compound, a carrier-drug compound is formed that can be blended with the adhesive compound to form the pressure-sensitive adhesive. In some embodiments, the adhesive compound comprises approximately 50 to 90 percent weight (w/w %) of the pressure-sensitive adhesive and the carrier-drug compound comprises approximately 10 to 50 w/w % of the pressure-sensitive adhesive.

The carrier-drug compound can also be melted, or dissolved with a solvent, to facilitate blending with the adhesive compound using the same conditions described above for the drug transport compound. Solvent can also be added to the adhesive compound to reduce its viscosity and facilitate blending with the carrier-drug compound. In some embodiments, the solvent used with the drug transport compound can be mixed with the adhesive compound prior to its mixing with the carrier-drug compound. In some embodiments, the solvent can be added to the adhesive compound in a concentration of approximately 33 to 67 w/w %. By way of example, the solvent can be added to the adhesive compound in a concentration of approximately 45 to 55 w/w %.

In cases in which one or more solvents are used in the creation of the pressure-sensitive adhesive, the solvent(s) are later evaporated from the blend to obtain the completed pressure-sensitive adhesive. In some embodiments, the blend is applied as a layer of adhesive to a substrate (as described below) and is then heated to evaporate the solvent(s). By way of example, the layer is applied so as to have a mass per area of approximately 1.9 to 2.4 grams per 100 square inches. In some embodiments, the layer is heated to a temperature of approximately 140 to 280° F. for approximately 3 to 10 minutes. Such heating can, for example, be performed by passing the coated substrate through an oven. Notably, this heating is not great enough to cause significant crosslinking within the drug transport compound (e.g., glycerol-sebacic acid prepolymer). Indeed, to achieve even light crosslinking, the drug transport compound would need to be exposed to an elevated temperature for many hours. As such, the drug transport compound does not polymerize during heating. The resulting pressure-sensitive adhesive is highly tacky and sticks well to living tissue. Once heating is completed, the substrate can be wound up to form a roll of material that can be stored for later processing. In some embodiments, a release liner can be applied to the pressure-sensitive adhesive prior to such rolling to protect it.

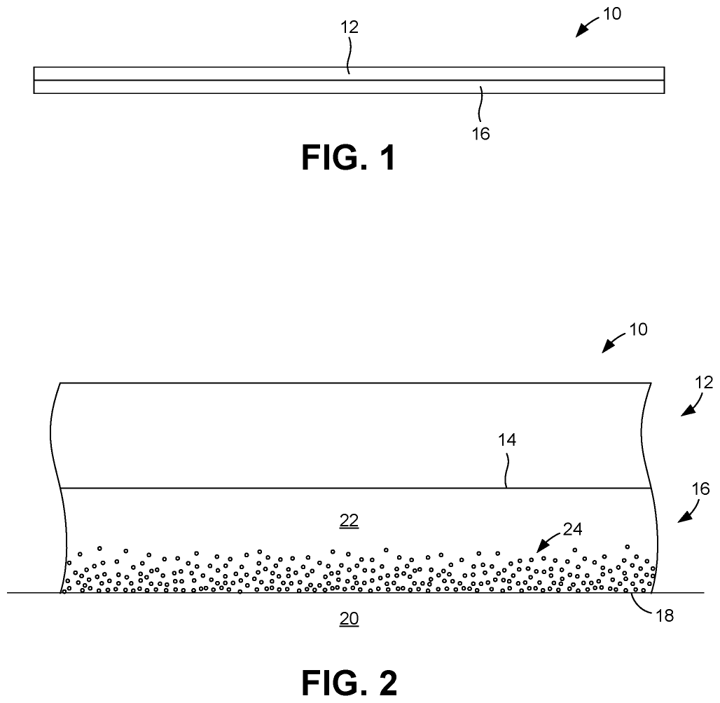

FIGS. 1 and 2 illustrate an embodiment of a transdermal patch 10 that incorporates the above-described pressure-sensitive adhesive. As shown in the figures, the patch 10 comprises a substrate 12 having an inner surface 14 to which has been applied a layer 16 of pressure-sensitive adhesive. The substrate 12 functions as a protective backing of the patch 10. In some embodiments, the substrate 12 comprises a flexible material that is adapted to conform to the contours of subjects to which the patch 10 is applied. Example constructions for the substrate 12 include one or more layers of paper, textile, polymer, foam, or foil. Example textiles include woven and nonwoven fabrics that are made of natural and/or manmade fibers. Example polymers include polyurethane, polypropylene, polyethylene, and polyvinyl chloride. In one specific embodiment, the substrate 12 is a non-woven polyurethane substrate.

The pressure-sensitive adhesive layer 16 can be approximately 10 to 200 μm thick. As shown in FIG. 2, the pressure-sensitive adhesive layer 16 forms an outer surface 18 that can be applied to the skin 20 when the transdermal patch 10 is used. As is also shown in FIG. 2, the pressure-sensitive adhesive layer 16 comprises an adhesive compound 22 in which discrete masses 24 of carrier-drug compound are dispersed.

As is apparent from FIG. 2, these masses 24 of carrier-drug compound have migrated toward the outer surface 18 and the skin 20 so as to deliver the drug or drugs to the patient. Such migration results from the immiscibility of the adhesive compound and the drug transport compound. Specifically, as the hydrophobic carrier-drug compound exists in small domains within the adhesive compound and is immiscible with the adhesive compound, the interfacial energy between the two compounds is high. This creates a driving force that causes the carrier-drug compound to separate from the adhesive compound, which causes the carrier-drug compound to gradually merge to form the masses 24 shown in FIG. 2. Because both the skin 20 and the carrier-drug compound are highly hydrophobic, the carrier-drug compound masses 24 naturally migrate toward the skin, as depicted in FIG. 2.

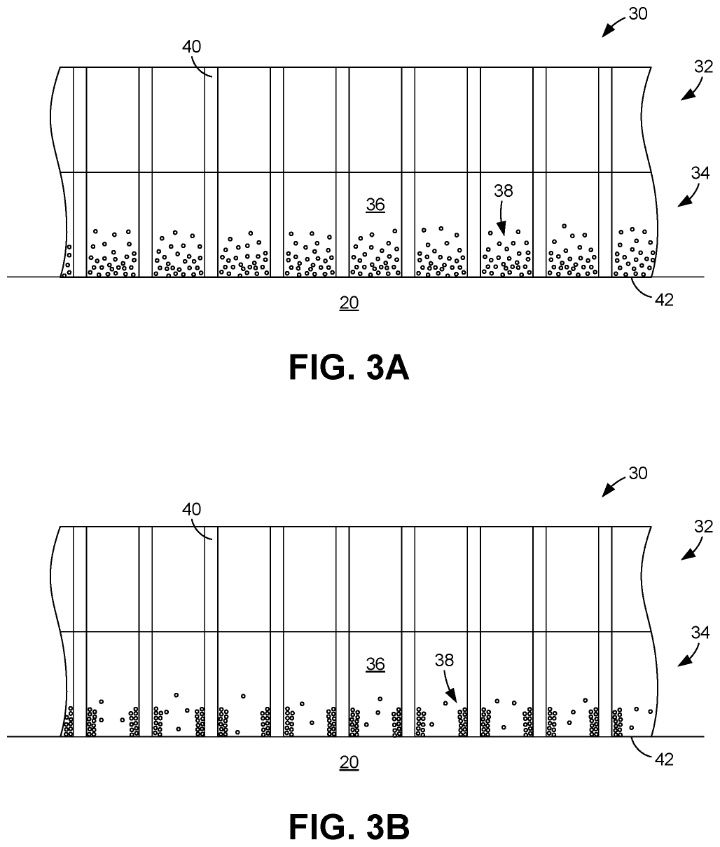

In some embodiments, this phenomenon can be augmented using an accumulation solution that further drives the carrier-drug compound toward the skin. In some embodiments, the accumulation solution can comprise an alcohol, such as isopropyl alcohol. An example of this is illustrated in FIG. 3A, which shows a further transdermal patch 30 that generally comprises a substrate 32 to which has been applied a layer 34 of pressure-sensitive adhesive. As with the previous patch 10, the pressure-sensitive adhesive comprises an adhesive compound 36 in which are dispersed masses 38 of carrier-drug compound, which are to be delivered to the surface of the skin 20. Perforations 40 are formed through the substrate 32 and the adhesive layer 34 that extend all the way to the outer surface 42 of the adhesive layer.

The accumulation solution can be applied to the patch 30 to drive the carrier-drug compound to the skin 20. FIG. 3B shows the patch 30 after the accumulation solution has been applied to the substrate 32 and has traveled down through the perforations 40 to the skin 20. When the solution reaches the skin 20, the solution collects at the interface between the adhesive layer 34 and the skin at the perforation sites. As depicted FIG. 3B, this collected solution attracts the carrier-drug compound masses 24 and therefore causes the masses to accumulate around the perforations 40 near the outer surface 42 of the adhesive layer 34, thereby placing even more carrier-drug compound in contact with the skin 20. In some embodiments, the solution can be applied to the patch 30 periodically to facilitate drug delivery to the skin 20.

When used, the accumulation solution can comprise approximately 50 to 90% alcohol. It is noted that the concentration of alcohol used in the solution can be selected to ensure accumulation of drug transport compound without negatively effecting the adhesive strength of the adhesive compound 36 or its bond with the skin 20 so as to avoid unintended release of the patch 30. For example, lower concentrations may result in the desired accumulation without unintended release. In addition, one or more of the size and number of perforations 40 can be tuned to ensure accumulation without unintended release.

Irrespective of whether or not an accumulation solution is used, greater drug delivery efficiency is obtained using the disclosed transdermal patches as compared to conventional transdermal patches. As used herein the term “drug delivery efficiency” refers to the percentage of drug initially provided in a patch that can actually be delivered to the skin. With the disclosed transdermal patches, much higher percentages of the drug initially provided in the patch can be delivered to the individual, thereby greatly increasing drug bioavailability and greatly reducing the amount of residual drug left in the patch. By way of example, the disclosed transdermal patches have a drug delivery efficiency of at least 70%, meaning that at least 70% of the drug initially provided in the patch (more particularly, in the pressure-sensitive adhesive of the patch) can be delivered to the individual and that no more than 30% of the drug initially provided in the patch remains as residual drug within the patch after its use. In some embodiments, the delivery efficiency is at least 80%, at least 90%, at least 95%, or at least 99%. In addition to being more efficient, the disclosed transdermal patches may also be used to deliver higher dosages and/or provide higher dosage rates than conventional transdermal patches.

It is noted that, in some embodiments, one or more additives can be added to the pressure-sensitive adhesive to improve skin permeation rates and/or comfort. Such additives can include one or more of dihydroxyaluminum aminoacetate, disodium edetate, gelatin, glycerin, kaolin, methylparaben, polyacrylic acid, polyvinyl alcohol, propylene glycol, propylparaben, sodium carboxymethylcellulose, sodium polyacrylate, D-sorbitol, tartaric acid, levulinic acid, lactic acid, ethyl oleate, and urea.

Claimed are:

1. A transdermal patch comprising:
a substrate; and
a layer of pressure-sensitive adhesive provided on the substrate, the pressure-sensitive adhesive comprising a blend of an adhesive compound and a carrier-drug compound, the carrier-drug compound comprising (a) a drug-transport compound configured to deliver one or more drugs to skin of a subject to which the transdermal patch is applied and (b) a drug;
wherein the drug-transport compound is a prepolymer formed from a multifunctional alcohol and a multifunctional carboxylic acid, and
wherein the drug-transport compound naturally migrates through the pressure-sensitive adhesive to the skin to deliver the drug to the subject's skin where it can be absorbed by the skin.

2. The patch of claim 1, wherein the pressure-sensitive adhesive comprises approximately 50 to 90 percent adhesive compound and approximately 10 to 50 percent carrier-drug compound by weight.

3. The patch of claim 1, wherein the adhesive compound comprises an acrylic polymer adhesive, a silicone adhesive, a rubber adhesive, a polyurethane adhesive, a hydrocolloid blended with an adhesive, or a mixture thereof.

4. The patch of claim 1, wherein the adhesive compound comprises an acrylic polymer adhesive.

5. The patch of claim 1, wherein the drug transport compound is hydrophobic.

6. The patch of claim 1, wherein the multifunctional alcohol comprises glycerol, a monomeric carbohydrate, a polyol, or a mixture thereof.

7. The patch of claim 1, wherein the multifunctional carboxylic acid comprises a diacid, a triacid, or a mixture thereof.

8. The patch of claim 1, wherein the drug transport compound comprises glycerol-sebacic acid prepolymer.

9. The patch of claim 1, wherein the drug comprises fentanyl, buprenorphine, aspirin, acetaminophen, lidocaine, bupivacaine, nitroglycerin, paclitaxel, estradiol, ethinyl estradiol, estrogen, nicotine, clonidine, fentanyl hcl, scopolamine, testosterone, epinephrine, norethindrone, norelgestromin, levonorgestrel, oxybutynin, tetracaine, methylphenidate, selegiline, rotigotine, rivastigmine, menthol, methyl salicylate, methyphenidate, vitamin B12, 5-Hydroxytryptophan, lorazepam, diphenhydramine, haloperidol, sumatriptan, stilboestrol, sufentanil, or a mixture thereof.

10. The patch of claim 1, further comprising perforations that extend through the substrate and the adhesive layer.

11. The patch of claim 10, further comprising an accumulation solution provided within the perforations that drives the carrier-drug compound to the skin.

12. The patch of claim 11, wherein the accumulation solution comprises an alcohol.

13. The patch of claim 1, wherein the patch has a drug delivery efficiency of at least 70 percent.

14. The patch of claim 1, wherein the patch has a drug delivery efficiency of at least 80 percent.

15. The patch of claim 1, wherein the patch has a drug delivery efficiency of at least 90 percent.

16. The patch of claim 1, wherein the patch has a drug delivery efficiency of at least 95 percent.
